(19) United States
(12) Patent Application Publication
(10) Pub. No.: US 2022/0015826 A1
(43) Pub. Date: Jan. 20, 2022

(54) Catheter and Method for Focal Cardiac Ablation by Irreversible Electroporation

(71) Applicant: Boston Scientific Scimed Inc., Maple Grove, MN (US)

(72) Inventors: Brendan E. Koop, Ham Lake, MN (US); Andrew L. De Kock, Ham Lake, MN (US); Allan C. Shuros, St Paul, MN (US); Edward J. Maierhofer, Brooklyn Park, MN (US)

(21) Appl. No.: 17/378,219

(22) Filed: Jul. 16, 2021

Related U.S. Application Data
(60) Provisional application No. 63/052,823, filed on Jul. 16, 2020.

(51) Int. Cl. A61B 18/14; A61M 25/00

(52) U.S. Cl. CPC A61B 18/1492; A61B 2018/00351; A61M 25/005

(57) ABSTRACT

A catheter for focal cardiac ablation by irreversible electroporation includes a flexible catheter body, a plurality of tines disposed at a distal end of the catheter body, a flexible shaft, a return electrode, and an electrical conductor. The plurality of tines are formed of an electrically conductive material and configured to deploy from a lumen at the distal end of the catheter body. Each tine of the plurality of tines is configured to self-bias from a linear configuration within the lumen to a curved configuration when deployed from the lumen. The shaft is mechanically and electrically coupled to the plurality of tines. The shaft is configured to deploy the tines from the lumen when the shaft is moved toward the distal end of the catheter body. The return electrode is disposed on an outer surface of the catheter body. The electrical conductor is electrically coupled to the return electrode.

### CROSS REFERENCE TO RELATED APPLICATION

This application claims priority to Provisional Application No. 63/052,823, filed Jul. 16, 2020, which is herein incorporated by reference in its entirety.

### TECHNICAL FIELD

The present invention relates to catheters and methods for the ablation of cardiac tissue. More specifically, the invention relates to catheters and methods for focal ablation of cardiac tissue by irreversible electroporation.

### BACKGROUND

Aberrant conductive pathways can disrupt the normal path of the heart's electrical impulses. The aberrant conductive pathways can create abnormal, irregular, and sometimes life-threatening heart rhythms called arrhythmias. Ablation of cardiac tissue is one way of treating arrhythmias and restoring normal conduction. The specific cardiac tissue can be located or mapped using mapping electrodes of a mapping catheter. After mapping, the physician may ablate the aberrant tissue.

Precision, point-by-point, or focal, cardiac ablation is generally accomplished using radio frequency (RF) energy. In radio frequency (RF) ablation, RF energy is directed from an ablation electrode through tissue to an electrode to ablate the tissue and form a lesion. RF energy destroys tissue indiscriminately through cell necrosis, which can result to damage to untargeted tissue, such as nerve and arterial tissues, for example. RF ablation can create other undesired effects, such as tissue charring and steam pops due to the heat generated by the RF energy.

### SUMMARY

Example 1 is a catheter for focal cardiac ablation by irreversible electroporation. The catheter includes a flexible catheter body extending from a proximal end to a distal end, a plurality of tines disposed at the distal end of the catheter body, a flexible shaft, a return electrode, and an electrical conductor. The catheter body forms a lumen extending from the proximal end to the distal end. The catheter body defining a longitudinal axis. The plurality of tines are formed of an electrically conductive material and configured to deploy from the lumen at the distal end of the catheter body. Each tine of the plurality of tines is configured to self-bias from a linear configuration within the lumen to a curved configuration when deployed from the lumen. The flexible shaft is formed of an electrically conductive material. The shaft is mechanically and electrically coupled to the plurality of tines and extends through the lumen from the proximal end of the catheter body. The shaft is configured to deploy the tines from the lumen when the shaft is moved toward the distal end of the catheter body. The return electrode is disposed on an outer surface of the catheter body. The electrical conductor is electrically coupled to the return electrode. The electrical conductor extends through the catheter body from the proximal end of the catheter body.

Example 2 is the catheter of Example 1, wherein the return electrode includes a ring extending around a circumference of the catheter body.

Example 3 is the catheter of Example 1, wherein the return electrode includes a first ring and a second ring, each of the first ring and the second ring extending around a circumference of the catheter body, the second ring disposed proximally from the first ring.

Example 4 is the catheter of Example 1, wherein the return electrode includes at least one of: a wire coil, a wire mesh and a hypo tube cut in a flexible pattern, the return electrode extending around a circumference of the catheter body.

Example 5 is the catheter of Example 1, wherein catheter body includes a deployable array at the distal end of the catheter body and the return electrode includes a plurality of return electrodes disposed on the deployable array, the deployable array including a plurality of splines, each spline of the plurality of splines including at least one return electrode of the plurality of return electrodes.

Example 6 is the catheter of Example 5, wherein the plurality of splines are configured to transition between a retracted configuration in which each spline of the plurality of splines is substantially parallel to the longitudinal axis and a deployed configuration in which each spline of the plurality of splines is bowed radially outward from the longitudinal axis.

Example 7 is the catheter of any of Examples 1-6, wherein the flexible shaft is formed of a wire coil.

Example 8 it the catheter of any of Example 1-7, wherein the return electrode is disposed between 1 mm and 10 mm from the distal end of the catheter body.

Example 9 is the catheter of any of Example 1-8, wherein each tine of the plurality of tines forms a curve extending from the distal end of the catheter body and away from the longitudinal axis in the curved configuration.

Example 10 is a catheter for focal cardiac ablation by irreversible electroporation. The catheter includes a flexible catheter body extending from a proximal end to a distal end, a plurality of tines disposed at the distal end of the catheter body, a flexible shaft, a return electrode and an electrical conductor. The catheter body forms a lumen extending from the proximal end to the distal end. The catheter body defines a longitudinal axis. The plurality of tines are formed of an electrically conductive material and are configured to deploy from the lumen at the distal end of the catheter body. Each tine of the plurality of tines is configured to self-bias from a linear configuration within the lumen to a curved configuration when deployed from the lumen. The flexible shaft is formed of an electrically conductive material. The shaft is mechanically and electrically coupled to the plurality of tines and extends through the lumen from the proximal end of the catheter body. The shaft is configured to deploy the tines from the lumen when the shaft is moved toward the distal end of the catheter body. The return electrode is configured to deploy from the catheter body at the distal end of the catheter body and to form a loop extending from the catheter body. The electrical conductor is electrically coupled to the return electrode. The electrical conductor extends through the catheter body from the proximal end of the catheter body.

Example 11 is the catheter of Example 10, wherein the return electrode includes a first wire coil.

Example 12 is the catheter of Example 11, wherein the electrical conductor and the return electrode each include the first wire coil.

Example 13 is the catheter of any of Examples 10-12, wherein the flexible shaft is formed of a second wire coil.

Example 14 is the catheter of any of Examples 10-13, wherein each tine of the plurality of tines forms a curve extending from the distal end of the catheter body and away from the longitudinal axis in the curved configuration.

Example 15 is the catheter of any of Examples 10-14, wherein the loop formed by the return electrode is configured to selectively rotate around the longitudinal axis.

Example 16 is a catheter for focal cardiac ablation by irreversible electroporation. The catheter includes a flexible catheter body extending from a proximal end to a distal end, a plurality of tines disposed at the distal end of the catheter body, a flexible shaft, a return electrode, and an electrical conductor. The catheter body forms a lumen extending from the proximal end to the distal end. The catheter body defining a longitudinal axis. The plurality of tines are formed of an electrically conductive material and configured to deploy from the lumen at the distal end of the catheter body Each tine of the plurality of tines is configured to self-bias from a linear configuration within the lumen to a curved configuration when deployed from the lumen. Each tine of the plurality of tines forms a curve extending from the distal end of the catheter body and away from the longitudinal axis in the curved configuration. The flexible shaft is formed of an electrically conductive material. The shaft is mechanically and electrically coupled to the plurality of tines and extends through the lumen from the proximal end of the catheter body. The shaft is configured to deploy the tines from the lumen when the shaft is moved toward the distal end of the catheter body. The return electrode is disposed on an outer surface of the catheter body. The electrical conductor is electrically coupled to the return electrode. The electrical conductor extends through the catheter body from the proximal end of the catheter body.

Example 17 is the catheter of Example 16, wherein the return electrode includes a ring extending around a circumference of the catheter body.

Example 18 is the catheter of Example 16, wherein the return electrode includes a first ring and a second ring, each of the first ring and the second ring extending around a circumference of the catheter body, the second ring disposed proximally from the first ring.

Example 19 is the catheter of Example 16, wherein the return electrode includes at least one of: a wire coil, a wire mesh and a hypo tube cut in a flexible pattern, the return electrode extending around a circumference of the catheter body.

Example 20 is the catheter of Example 16, wherein catheter body includes a deployable array at the distal end of the catheter body and the return electrode includes a plurality of return electrodes disposed on the deployable array, the deployable array including a plurality of splines, each spline of the plurality of splines including at least one return electrode of the plurality of return electrodes.

Example 21 is the catheter of Example 20, wherein the plurality of splines are configured to transition between a retracted configuration in which each spline of the plurality of splines is substantially parallel to the longitudinal axis and a deployed configuration in which each spline of the plurality of splines is bowed radially outward from the longitudinal axis.

Example 22 is the catheter of any of Examples 16-21, wherein the flexible shaft is formed of a wire coil.

Example 23 is the catheter of any of Examples 16-22, wherein the return electrode is disposed between 1 mm and 10 mm from the distal end of the catheter body.

Example 24 is the catheter of any of Examples 16-23, further comprising an atraumatic tip disposed at the at the distal end of the catheter body.

Example 25 is a catheter for focal cardiac ablation by irreversible electroporation. The catheter includes a flexible catheter body extending from a proximal end to a distal end, a plurality of tines disposed at the distal end of the catheter body, a flexible shaft, a return electrode and an electrical conductor. The catheter body forms a lumen extending from the proximal end to the distal end. The catheter body defines a longitudinal axis. The plurality of tines are formed of an electrically conductive material and are configured to deploy from the lumen at the distal end of the catheter body. Each tine of the plurality of tines is configured to self-bias from a linear configuration within the lumen to a curved configuration when deployed from the lumen. The flexible shaft is formed of an electrically conductive material. The shaft is mechanically and electrically coupled to the plurality of tines and extends through the lumen from the proximal end of the catheter body. The shaft is configured to deploy the tines from the lumen when the shaft is moved toward the distal end of the catheter body. The return electrode is configured to deploy from the catheter body at the distal end of the catheter body and to form a loop extending from the catheter body. The electrical conductor is electrically coupled to the return electrode. The electrical conductor extends through the catheter body from the proximal end of the catheter body.

Example 26 is the catheter of Example 25, wherein the return electrode includes a first wire coil.

Example 27 is the catheter of Example 26, wherein the electrical conductor and the return electrode each include the first wire coil.

Example 28 is the catheter of any of Examples 25-27, wherein the flexible shaft is formed of a second wire coil.

Example 29 is the catheter of any of Examples 25-28, wherein each tine of the plurality of tines forms a curve extending from the distal end of the catheter body and away from the longitudinal axis in the curved configuration.

Example 30 is the catheter of any of Examples 25-29, wherein the loop formed by the return electrode is configured to selectively rotate around the longitudinal axis.

Example 31 is the catheter of any of Examples 25-30, further comprising an atraumatic tip disposed at the at the distal end of the catheter body.

Example 32. A method for focal cardiac ablation by irreversible electroporation. The method includes inserting a distal end of a catheter into a heart and adjacent to tissue containing cells to be ablated, the catheter including a flexible catheter body and a plurality of electrically conductive tines configured to deploy from a lumen at the distal end of the catheter body; moving a shaft extending through the lumen toward the distal end to deploy the plurality of electrically conductive tines from the lumen and into the tissue containing the cells to be ablated, each tine of the plurality of electrically conductive tines self-biasing from a linear configuration within the lumen to a curved configuration when deployed into the tissue; applying a series of voltage pulses between the plurality of electrically conductive tines and a return electrode disposed on an outer surface of the catheter body and spaced apart from the distal end of the catheter body, the voltage pulses forming an electric field causing irreversible electroporation of the cells to be ablated; moving the shaft extending through the lumen toward a proximal end of the catheter body to retract the tines into the lumen; and withdrawing the distal end of the catheter from the heart.

Example 33 is the method of Example 32, wherein the return electrode is disposed between 1 mm and 10 mm from the distal end of the catheter body.

Example 34 is the method of Example 32 or Example 33, wherein each tine of the plurality of tines forms a curve extending from the distal end of the catheter body and away from a longitudinal axis of the catheter body in the curved configuration.

Example 35 is the method of any of Examples 32-34, wherein the return electrode includes a plurality of electrodes, the method further including deploying an array including a plurality of splines at the distal end of the catheter body by transitioning from a retracted configuration in which each spline of the plurality of splines is substantially parallel to a longitudinal axis of the catheter body and a deployed configuration in which each spline of the plurality of splines is bowed radially outward from the longitudinal axis of the catheter body, each spline of the plurality of splines including at least one return electrode of the plurality of return electrodes, wherein deploying the array is after inserting the distal end of a catheter into the heart and \and before applying the series of voltage pulses; and retracting the array by transitioning from the deployed configuration to the retracted configuration after applying the series of voltage pulses and before withdrawing the distal end of the catheter from the heart.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A and 1B are schematic perspective views of a distal end of a catheter for focal cardiac ablation by irreversible electroporation in undeployed and deployed configurations, respectively, according to some embodiments of this disclosure.

FIG. 2 is a schematic side view of the distal end of a catheter for focal cardiac ablation by irreversible electroporation deployed into tissue, according to some embodiments of this disclosure.

FIG. 3 is a schematic side view of the distal end of another catheter for focal cardiac ablation by irreversible electroporation deployed into tissue, according to some embodiments of this disclosure.

FIG. 4 is a schematic side view of the distal end of yet another catheter for focal cardiac ablation by irreversible electroporation deployed into tissue, according to some embodiments of this disclosure.

FIGS. 5A and 5B are schematic perspective views of a distal end of a catheter for focal cardiac ablation by irreversible electroporation in undeployed and deployed configurations, respectively, according to some other embodiments of this disclosure.

FIGS. 6A and 6B are schematic perspective views of a distal end of a catheter for focal cardiac ablation by irreversible electroporation in undeployed and deployed configurations, respectively, according to some other embodiments of this disclosure.

While the disclosure is amenable to various modifications and alternative forms, specific embodiments have been shown by way of example in the drawings and are described in detail below. The intention, however, is not to limit the invention to the particular embodiments described. On the contrary, the disclosure is intended to cover all modifications, equivalents, and alternatives falling within the scope of the invention as defined by the appended claims.

### DETAILED DESCRIPTION

Irreversible electroporation uses trains of short, high voltage pulses to kill cells by forming lethal nanopores in the cell membranes. The damaged cells then die through apoptosis. The voltage pulses of irreversible electroporation can be targeted to kill myocardium, and leave other tissues relatively unscathed, thus avoiding the undesired side effects of RF ablation. However, forming transmural lesions in thick myocardium can be difficult with irreversible electroporation. In addition, ablation of heart tissue poses a challenge in that the heart is constantly moving during an ablation procedure. As a result, it can be difficult to maintain stable contact between an ablating electrode and the target tissue. Embodiments of the present disclosure provide catheters and methods for focal cardiac ablation by irreversible electroporation that reduce these problems.

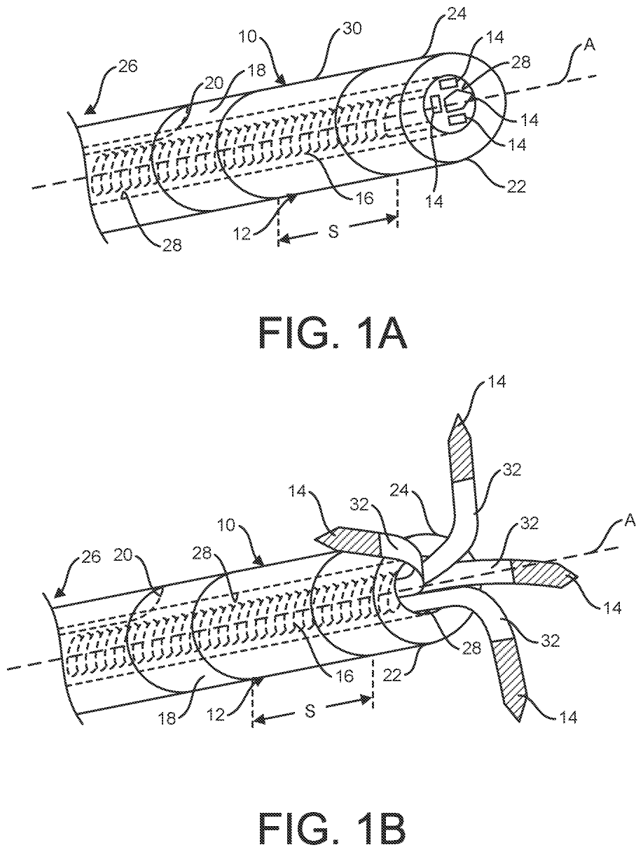

FIGS. 1A and 1B are schematic perspective views of a distal end of a catheter for focal cardiac ablation by irreversible electroporation, according to some embodiments of this disclosure. FIG. 1A shows a catheter 10 including flexible catheter body 12, a plurality of tines 14 (four shown), a flexible shaft 16, a return electrode 18, and an electrical conductor 20. In some embodiments, the catheter 10 may further include an atraumatic tip 22, as shown in FIG. 1A. The catheter body 12 extends from a proximal end 26 to a distal end 24. The catheter body 12 forms a lumen 28 extending from the proximal end 26 to the distal end 24. The catheter body 12 defines a longitudinal axis A. The catheter body 12 may be formed of a flexible, insulative, biocompatible material known in the art, such as polyisobutylene polyurethane, silicone or polyether block amide, for example. The catheter body 12 may further include a metallic braiding.

The flexible shaft 16 includes an electrically conductive biocompatible material, such as a platinum iridium alloy, gold, stainless-steel, a titanium alloy, or a nickel-cobalt alloy, such as MP35N, for example. The flexible shaft 16 may further include an insulating polymer coating. In some embodiments, the flexible shaft 16 is in the form of a wire coil. The electrical conductor 20 is also formed of any of the previously mentioned electrically conductive materials and may also further include an insulating polymer coating.

The optional atraumatic tip 22 may formed of a biocompatible material, such as polyether ether ketone (PEEK), polyisobutylene polyurethane, silicone, polyether block amide or titanium, for example. The atraumatic tip 22 is disposed at the distal end 24 and forms the tip of the catheter 10 when the catheter 10 is in the undeployed configuration, as shown in FIG. 1A.

The plurality of tines 14 is disposed at the distal end 24 of the catheter body 12. The flexible shaft 16 is mechanically and electrically coupled to the plurality of tines 14 and extends through the lumen 28 from the proximal end 26 of the catheter body 12. FIG. 1A shows the plurality of tines 14 in an undeployed configuration, with each tine of the plurality of tines 14 substantially contained within the lumen 28.

The return electrode 18 is disposed on an outer surface 30 of the catheter body 12. In the embodiment shown in FIG. 1A, the return electrode 18 includes a ring extending around a circumference of the catheter body 12.

The return electrode 18 is spaced apart from the distal end 24 of the catheter body 12 by a distance S, which may be as little as 1 mm, 2 mm, 3 mm, 4 mm or 5 mm, or as much as 6 mm, 7 mm, 8 mm, 9 mm or 10 mm, or within any range defined between any two of the foregoing values, such as 1 mm to 10 mm, 2 mm to 9 mm, 3 mm to 8 mm, 4 mm to 7 mm, 5 mm to 6 mm, 1 mm to 5 mm, 6 mm to 10 mm, 2 mm to 5 mm, 3 mm to 4 mm or 2 mm to 6 mm, for example.

The electrical conductor 20 is electrically coupled to the return electrode 18. The electrical conductor 20 extends through the catheter body 12 from the proximal end 26. In some embodiments, the electrical conductor 20 extends through the lumen 28 from the proximal end 26 of the catheter body 12, as shown in FIG. 1A. In some other embodiments, the electrical conductor 20 may extend from the proximal end 26 through a separate lumen (not shown) formed by the catheter body 12. In some other embodiments, the electrical conductor 20 may molded or formed into the catheter body

The flexible shaft 16 may be moved distally to deploy the plurality of tines 14 from the lumen 28, as shown in FIG. 1B. The plurality of tines 14 are formed of an electrically conductive material having a shape memory, for example Nitinol or a gold/stainless steel alloy, such that each tine of the plurality of tines 14 can biased to be curved configuration when unrestrained and outside of the lumen 28 as shown in FIG. 1B, and can be in a linear configuration when restrained within the lumen 28, as shown in FIG. 1A. Each tine of the plurality of tines 14 can be pointed for ease in entering tissue. In the curved configuration shown in FIG. 1B, each tine of the plurality of tines 14 forms a curve extending from the distal end 24 of the catheter body 12 and away from the longitudinal axis A.

As shown in FIG. 1B, each tine of the plurality of tines 14 may further include an insulating layer 32. The insulating layer 32 may be useful to prevent applying a voltage to the atraumatic tip 22 if the atraumatic tip 22 is formed of a conductive material. The insulating layer 32 may also be useful in some embodiments in which the flexible shaft 16 is in the form of a multifilar coil with each filar electrically connected to a different tine of the plurality of tines 14 so that they may be used in mapping the heart prior to the focal cardiac ablation. During such mapping, the plurality of tines 14 are deployed without penetrating tissue.

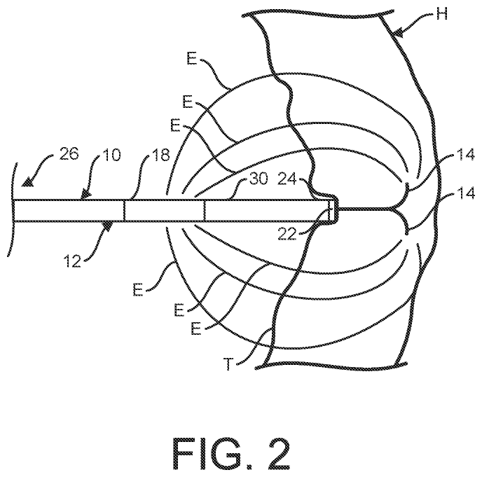

FIG. 2 is a schematic side view of the distal end 24 of the catheter 10 deployed into tissue, according to some embodiments of this disclosure. Considering FIGS. 1A, 1B and 2 together, in use, the distal end 24 of the catheter body 12 of the catheter 10 is inserted into a heart H and adjacent to tissue T containing cells to be ablated. The flexible shaft 16 (FIGS. 1A and 1B) is moved toward the distal end 24 to deploy plurality of tines 14 from the lumen 28 and into the tissue T. As each tine of the plurality of tines 14 moves out of the lumen 28 they transition from the liner configuration of FIG. 1A to the curved configuration shown in FIG. 1B. Each tine of the plurality of tines 14 penetrates the tissue T and, unconstrained by the catheter body 12, self-biases to the curved configuration outside of the lumen 28 as it penetrates through the tissue T, carving a curved path through the tissue T. So deployed, the plurality of tines 14 stabilizes the catheter 10 at the desired location before, during and after ablation. Once deployed, a series of voltage pulses is applied between the plurality of tines 14 and the return electrode 18 to form an electric field E of sufficient strength to cause irreversible electroporation of the cells of the tissue T to be ablated. Various sizes and deployment depths of the plurality of tines 14 can be used to target specific portions of the tissue T. The distal end 24 of the catheter body 12 does not penetrate into the tissue T.

Each tine of the plurality of tines 14 may penetrate tissue T to a depth of as little as 1 mm, 1.5 mm, 2 mm, 2.5 mm, 3 mm or 3.5 mm or as great as 4 mm, 4.5 mm, 5 mm, 5.5 mm, 6 mm, 6.5 mm or 7 mm, or within any range defined between any two of the foregoing values, such as 1 mm to 7 mm, 1.5 mm to 6.5 mm, 2 mm to 6 mm, 2.5 mm to 5.5 mm, 3 mm to 5 mm, 4 mm to 4.5 mm, 1 mm to 2 mm, 2 mm to 7 mm or 1 mm to 3 mm, for example.

In some embodiments, each tine of the plurality of tines 14 may have a rectangular cross-section having a cross-sectional length and a cross-sectional width. The cross-sectional length may be as small 0.25 mm, 0.30 mm, 0.35 mm, 0.40 mm, 0.45 mm or 0.50 mm, or as large as 0.55 mm, 0.60 mm, 0.65 mm, 0.70 mm, 0.75 mm or 0.80 mm, or within any range defined between any two of the foregoing values, such as 0.25 mm to 0.80 mm, 0.30 mm to 0.75 mm, 0.35 mm to 0.70 mm, 0.40 mm to 0.65 mm, 0.45 mm to 0.60 mm, 0.50 mm to 0.55 mm, 0.30 mm to 0.60 mm, 0.25 mm to 0.50 mm, or 0.55 mm to 0.75 mm, for example. The cross-sectional width may be as small 0.10 mm, 0.13 mm, 0.15 mm, 0.18 mm, 0.20 mm or 0.23 mm, or as large as 0.25 mm, 0.28 mm, 0.30 mm, 0.33 mm, 0.36 mm or 0.38 mm, or within any range defined between any two of the foregoing values, such as 0.10 mm to 0.38 mm, 0.13 mm to 0.36 mm, 0.15 mm to 0.33 mm, 0.18 mm to 0.30 mm, 0.20 mm to 0.28 mm, 0.23 mm to 0.25 mm, 0.20 mm to 0.30 mm, 0.10 mm to 0.20 mm, or 0.25 mm to 0.38 mm, for example.

The electric field E may be as low as 300 V/cm, 350 V/cm, 400 V/cm, 450 V/cm, 500 V/cm, 550 V/cm or 600 V/cm, or as high as 650 V/cm, 700 V/cm, 750 V/cm, 800 V/cm, 850 V/cm, 900 V/cm, 950 V/cm or 1,000 V/cm or within any range defined between any two of the foregoing values, such as 300 V/cm to 1,000 V/cm, 350 V/cm to 950 V/cm, 400 V/cm to 900 V/cm, 450 V/cm to 850 V/cm, 500 V/cm to 800 V/cm, 550 V/cm to 750 V/cm, 600 V/cm to 700 V/cm, 300 V/cm to 650 V/cm, 400 V/cm to 500 V/cm, or 700 V/cm to 900 V/cm, for example.

It is believed that the electric field E can be concentrated further into the depth of the tissue T to form transmural lesions in thick myocardium by deploying the plurality of tines 14 deep within the tissue T and generating the electric field between the plurality of tines 14 and the return electrode 18 disposed on the outer surface 30 of the catheter body 12 and spaced apart from the distal end 24.

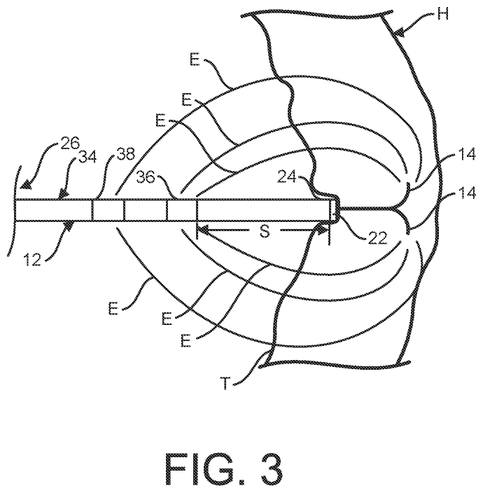

FIG. 3 is a schematic side view of the distal end 24 of another catheter for focal cardiac ablation by irreversible electroporation deployed into tissue, according to some embodiments of this disclosure. FIG. 3 shows a catheter 34. The catheter 34 is substantially identical to the catheter 10 described above, except that the single ring of the return electrode 18 as shown the embodiments of FIGS. 1A, 1B and 2, is replaced with a first ring 36 and a second ring 38, each ring extending around the circumference of the catheter body 12, the second ring 38 disposed proximally from the first ring 36. In some embodiments, the electrical conductor 20 (FIGS. 1A and 1B) is electrically connected to both the first ring 36 and the second ring 38. In this way, the electric field E may be shaped for more effective ablation of the tissue T. In some other embodiments, the electrical conductor 20 (FIGS. 1A and 1B) includes two separate conductors (not shown), electrically isolated from each other and each conductor electrically connected to the first ring 36 or the second ring 38. In this way, the strength and size of the electrical field E may be tuned to provide more targeted ablation of the tissue T. While the return electrode of the embodiment shown in FIG. 3 includes two rings (the first ring 36 and the second ring 38), it is understood that the disclosure includes embodiments having a return electrode including more than two rings.

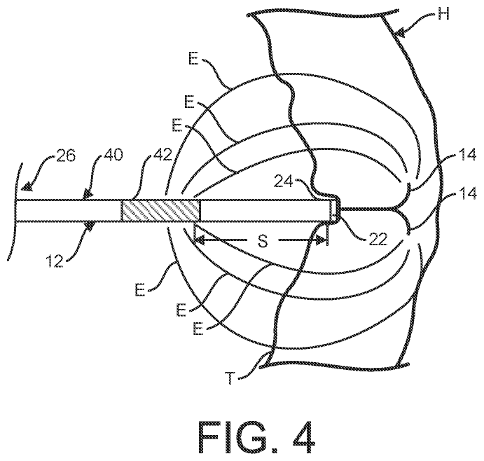

FIG. 4 is a schematic side view of the distal end 24 of yet another catheter for focal cardiac ablation by irreversible electroporation deployed into tissue, according to some embodiments of this disclosure. FIG. 4 shows a catheter 40. The catheter 40 is substantially identical to the catheter 10 described above, except that the single ring of the return electrode 18 as shown the embodiments of FIGS. 1A, 1B and 2, is replaced with a wire coil 42 extending around the circumference of the catheter body 12. The wire coil 42 may be more flexible than the single ring of the return electrode of FIGS. 1A, 1B and 2 described above, and may thus be longer without significantly decreasing the flexibility of the catheter body 12. The longer return electrode provided by the wire coil 42 may shape the electric field E for more effective ablation of the tissue T. While in some embodiments, the wire coil 42 is in the form of a coil, in some other embodiments, the wire coil 42 is in the form of a mesh or a hypo tube cut in a flexible pattern.

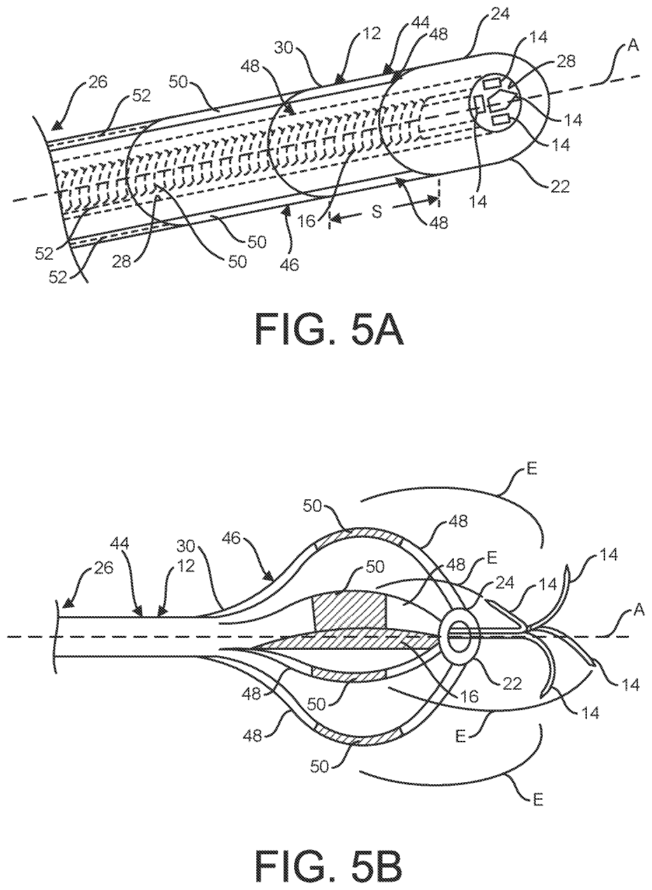

FIGS. 5A and 5B are schematic perspective views of a distal end 24 of a catheter for focal cardiac ablation by irreversible electroporation in undeployed and deployed configurations, respectively, according to some other embodiments of this disclosure. FIG. 5A shows a catheter 44 in the undeployed configuration. The catheter 44 is substantially identical to the catheter 10 described above, except that the catheter body 12 further includes a deployable array 46 including a plurality of splines 48, the return electrode 18 is replaced with a plurality of return electrodes 50, and the electrical conductor 20 is replaced with a plurality of electrical conductors 52. Each spline of the plurality of splines 48 includes at least one return electrode of the plurality of electrodes 50. Each return electrode of the plurality of return electrodes 50 is electrically coupled to at least one of electrical conductors 52 of the plurality of electrical conductors 52. Each electrical conductor of the plurality of electrical conductors 52 may be substantially identical to the electrical conductor 20 described above. Although the catheter 44 includes four splines 48 (see FIG. 5B), only three are visible in FIG. 5A.

In some embodiments, the plurality of splines 48 are formed of the same material making up the rest of the catheter body 12, such polyether block amide, polyisobutylene polyurethane or silicone. In some embodiments, the plurality of splines 48 may further include a material having a shape memory, for example Nitinol or a gold/stainless steel alloy.

The plurality of splines 48 are configured to transition between an undeployed configuration shown in FIG. 5A, to a deployed configuration in FIG. 5B. The heart H and tissue T are omitted for clarity of illustration. As shown in FIG. 5A, in the undeployed configuration, each spline of the plurality of splines 48 is substantially parallel to the longitudinal axis A. As shown in FIG. 5B, in the deployed configuration, each spline of the plurality of splines 48 is bowed radially outward from the longitudinal axis. Deploying the deployable array 46 may occur before, after or coincidental with deploying the plurality of tines 14, as describe above in reference to FIG. 2. In this way, the strength and size of the electrical field E may be tuned and the electrical field # steered to provide more targeted ablation of the tissue T.

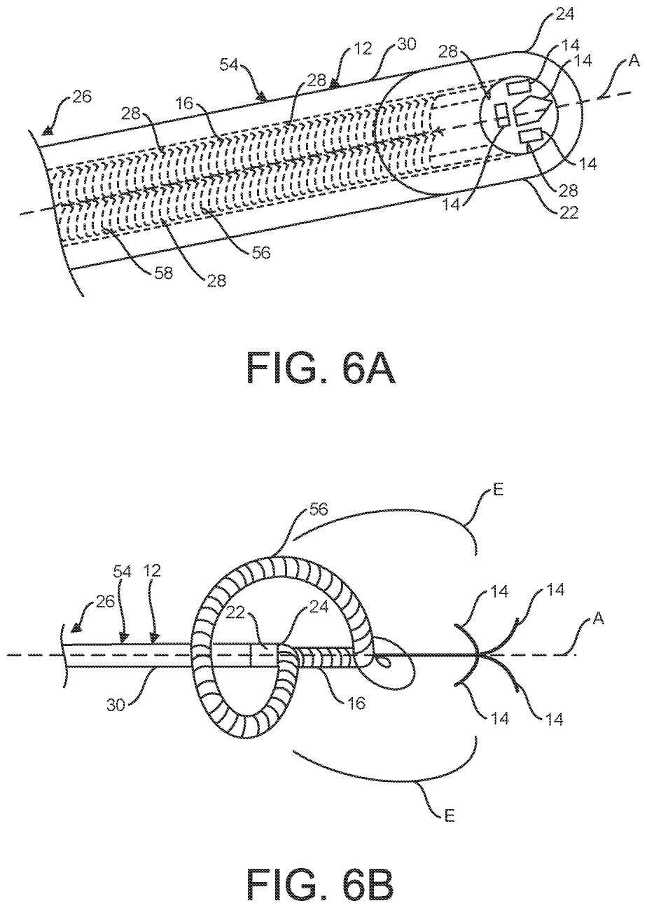

FIGS. 6A and 6B are schematic perspective views of a distal end 24 of a catheter for focal cardiac ablation by irreversible electroporation in undeployed and deployed configurations, respectively, according to some other embodiments of this disclosure. FIG. 6A shows a catheter 54 in the undeployed configuration. The catheter 54 is substantially identical to the catheter 10 described above, except that the return electrode 18 is replaced with a return electrode 56 and the electrical conductor 20 is replace with an electrical conductor 58. Unlike the embodiments describe above, the return electrode 56 is not disposed on the outer surface 30 of the catheter body 12.

The return electrode 56 is formed of an electrically conductive biocompatible material, such as stainless steel, platinum, platinum iridium alloy, platinum-clad tantalum, titanium, Nitinol, or a nickel-cobalt alloy, such as MP35N, for example. In some embodiments, the return electrode 56 is in the form of a wire coil. In some other embodiments, the return electrode 56 is in the form of a mesh or a hypo tube cut in a flexible pattern. The electrical conductor 58 is also formed of an electrically conductive material, such as stainless steel, platinum, platinum iridium alloy, platinum-clad tantalum, titanium, Nitinol, or a nickel-cobalt alloy, such as MP35N, for example. In some embodiments, the electrical conductor 58 is in the form of a wire coil. In some other embodiments, the electrical conductor 58 is in the form of a mesh or a hypo tube cut in a flexible pattern. In some embodiments, the return electrode 56 and the electrical conductor 58 may form a single, continuous wire coil. In embodiments in which the return electrode 56 and the flexible shaft 16 are both formed of different wire coils within the catheter body 12, the wire coil of the return electrode 56 may be a first coil and the wire coil of the flexible shaft 16 may be a second coil.

FIG. 6B shows the catheter 54 in the deployed configuration. As shown in FIG. 6B, the return electrode 56 is configured to deploy from the lumen 28 at the distal end 24. As with FIG. 5B, the heart H and tissue T are omitted for clarity of illustration. The deployed return electrode 56 is configured to form a loop extending from the lumen 28. In some embodiments, articulation of the return electrode 56 may be controlled by extending and retracting the return electrode 56 from the lumen 28. In some embodiments, articulation of the return electrode 56 may be controlled by a pull wire (not shown). In some embodiments, the return electrode 56 is configured to selectively rotate around the longitudinal axis A. By rotating the return electrode 56 around the longitudinal axis A, the loop formed by the return electrode 56 moves relative to the plurality of tines 14, changing the position of the electrical field E. By extending and retracting the return electrode 56 from the lumen 28, the size of the loop may also be changed. In this way, the strength and position of the electrical field E may be tuned to provide more targeted ablation of the tissue T.

For clarity of illustration, all embodiments described above are shown with the plurality of tines 14 including four tines. However, it is understood that the disclosure encompasses embodiments with as few as two tines and as many as six tines.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

We claim:

1. A catheter for focal cardiac ablation by irreversible electroporation, the catheter comprising:
a flexible catheter body extending from a proximal end to a distal end, the catheter body forming a lumen extending from the proximal end to the distal end, the catheter body defining a longitudinal axis;
a plurality of tines disposed at the distal end of the catheter body, the plurality of tines formed of an electrically conductive material and configured to deploy from the lumen at the distal end of the catheter body, each tine of the plurality of tines configured to self-bias from a linear configuration within the lumen to a curved configuration when deployed from the lumen, each tine of the plurality of tines forming a curve extending from the distal end of the catheter body and away from the longitudinal axis in the curved configuration;
a flexible shaft formed of an electrically conductive material, the shaft mechanically and electrically coupled to the plurality of tines and extending through the lumen from the proximal end of the catheter body, the shaft configured to deploy the tines from the lumen when the shaft is moved toward the distal end of the catheter body;
a return electrode disposed on an outer surface of the catheter body; and
an electrical conductor electrically coupled to the return electrode, the electrical conductor extending through the catheter body from the proximal end of the catheter body.

2. The catheter of claim 1, wherein the return electrode includes a ring extending around a circumference of the catheter body.

3. The catheter of claim 1, wherein the return electrode includes a first ring and a second ring, each of the first ring and the second ring extending around a circumference of the catheter body, the second ring disposed proximally from the first ring.

4. The catheter of claim 1, wherein the return electrode includes at least one of: a wire coil, a wire mesh and a hypo tube cut in a flexible pattern, the return electrode extending around a circumference of the catheter body.

5. The catheter of claim 1, wherein catheter body includes a deployable array at the distal end of the catheter body and the return electrode includes a plurality of return electrodes disposed on the deployable array, the deployable array including a plurality of splines, each spline of the plurality of splines including at least one return electrode of the plurality of return electrodes.

6. The catheter of claim 5, wherein the plurality of splines are configured to transition between a retracted configuration in which each spline of the plurality of splines is substantially parallel to the longitudinal axis and a deployed configuration in which each spline of the plurality of splines is bowed radially outward from the longitudinal axis.

7. The catheter of claim 1, wherein the flexible shaft is formed of a wire coil.

8. The catheter of claim 1, wherein the return electrode is disposed between 1 mm and 10 mm from the distal end of the catheter body.

9. The catheter of claim 1, further comprising an atraumatic tip disposed at the at the distal end of the catheter body.

10. A catheter for focal cardiac ablation by irreversible electroporation, the catheter comprising:
a flexible catheter body extending from a proximal end to a distal end, the catheter body forming a lumen extending from the proximal end to the distal end, the catheter body defining a longitudinal axis;
a plurality of tines disposed at the distal end of the catheter body, the plurality of tines formed of an electrically conductive material and configured to deploy from the lumen at the distal end of the catheter body, each tine of the plurality of tines configured to self-bias from a linear configuration within the lumen to a curved configuration when deployed from the lumen;
a flexible shaft formed of an electrically conductive material, the shaft mechanically and electrically coupled to the plurality of tines and extending through the lumen from the proximal end of the catheter body, the shaft configured to deploy the tines from the lumen when the shaft is moved toward the distal end of the catheter body;
a return electrode configured to deploy from the catheter body at the distal end of the catheter body and to form a loop extending from the catheter body; and
an electrical conductor electrically coupled to the return electrode, the electrical conductor extending through the catheter body from the proximal end of the catheter body.

11. The catheter of claim 10, wherein the return electrode includes a first wire coil.

12. The catheter of claim 11, wherein the electrical conductor and the return electrode each include the first wire coil.

13. The catheter of claim 10, wherein the flexible shaft is formed of a second wire coil.

14. The catheter of claim 10, wherein each tine of the plurality of tines forms a curve extending from the distal end of the catheter body and away from the longitudinal axis in the curved configuration.

15. The catheter of claim 10, wherein the loop formed by the return electrode is configured to selectively rotate around the longitudinal axis.

16. The catheter of claim 10, further comprising an atraumatic tip disposed at the at the distal end of the catheter body.

17. A method for focal cardiac ablation by irreversible electroporation, the method comprising:
inserting a distal end of a catheter into a heart and adjacent to tissue containing cells to be ablated, the catheter including a flexible catheter body and a plurality of electrically conductive tines configured to deploy from a lumen at the distal end of the catheter body;
moving a shaft extending through the lumen toward the distal end to deploy the plurality of electrically conductive tines from the lumen and into the tissue containing the cells to be ablated, each tine of the plurality of electrically conductive tines self-biasing from a linear configuration within the lumen to a curved configuration when deployed into the tissue;
applying a series of voltage pulses between the plurality of electrically conductive tines and a return electrode disposed on an outer surface of the catheter body and spaced apart from the distal end of the catheter body, the voltage pulses forming an electric field causing irreversible electroporation of the cells to be ablated;
moving the shaft extending through the lumen toward a proximal end of the catheter body to retract the tines into the lumen; and
withdrawing the distal end of the catheter from the heart.

18. The method of claim 17, wherein the return electrode is disposed between 1 mm and 10 mm from the distal end of the catheter body.

19. The method of claim 17, wherein each tine of the plurality of tines forms a curve extending from the distal end of the catheter body and away from a longitudinal axis of the catheter body in the curved configuration.

20. The method of claim 17 wherein the return electrode includes a plurality of electrodes, the method further comprising:
deploying an array including a plurality of splines at the distal end of the catheter body by transitioning from a retracted configuration in which each spline of the plurality of splines is substantially parallel to a longitudinal axis of the catheter body and a deployed configuration in which each spline of the plurality of splines is bowed radially outward from the longitudinal axis of the catheter body, each spline of the plurality of splines including at least one return electrode of the plurality of return electrodes, wherein deploying the array is after inserting the distal end of a catheter into the heart and \and before applying the series of voltage pulses; and
retracting the array by transitioning from the deployed configuration to the retracted configuration after applying the series of voltage pulses and before withdrawing the distal end of the catheter from the heart.
